(19) United States
(12) Patent Application Publication
(10) Pub. No.: US 2026/0097130 A1
(43) Pub. Date: Apr. 9, 2026

(54) METHOD FOR TREATING NEURODEGENERATIVE DISEASES

(71) Applicant: Suntec Medical, Inc., Walnut, CA (US)

(72) Inventors: Chun-Ting Cheng, Taipei (TW); Kuo-Liang Hou, Taipei City (TW); Pauline Ying Lau, Walnut, CA (US)

(21) Appl. No.: 19/406,217

(22) Filed: Dec. 2, 2025

Related U.S. Application Data
(63) Continuation of application No. PCT/US2024/032621, filed on Jun. 5, 2024.
(60) Provisional application No. 63/506,303, filed on Jun. 5, 2023.

(51) Int. Cl. A61K 47/68; A61K 9/51; A61K 31/353; A61K 47/69; C07K 16/28

(52) U.S. Cl. CPC A61K 47/6849; A61K 9/5123; A61K 9/5146; A61K 31/353; A61K 47/6803; A61K 47/6935; C07K 16/2803; C07K 16/2809

(57) ABSTRACT

The present invention provides a method of treating neurodegenerative diseases. The method comprises the step of administering to a subject in need thereof an effective amount of a polymer-flavonoid conjugate, or a nanocomplex having an outer shell comprising one or more polymer-flavonoid conjugates and optionally an inner shell comprising one or more flavonoid oligomer and a drug such as anti-CD3 or anti-CD33 encapsulated within the shells. The present method brings therapeutic effective materials through blood-brain barrier to treat neurodegenerative diseases. The present method is effective to treat neurodegenerative diseases such as Alzheimer's disease, Parkinson's disease, Lewy body dementia and Huntington's disease.

### CROSS-REFERENCE TO RELATED APPLICATION(S)

This application is a continuation of PCT/US2024/032621, filed Jun. 5, 2024; which claims the benefit of U.S. Provisional Application No. 63/506,303, filed Jun. 5, 2023. The contents of the above-identified applications are incorporated herein by reference in their entireties.

### FIELD OF THE INVENTION

The present invention provides methods of treating or preventing the relapse of symptoms of neurodegenerative diseases or. The method comprises the step of administering to a subject in need thereof an effective amount of a polymer-flavonoid conjugate; or nanocomplexes having a shell comprising one or more polymer-flavonoid conjugates or one or more flavonoid oligomers, or the combination thereof, and having an antibody drug encapsulated within the shells.

### BACKGROUND OF THE INVENTION

A neurodegenerative disease is caused by the progressive loss of structure or function of neurons, in the process known as neurodegeneration. Neurodegenerative diseases are a group of diseases which primarily affect the neurons in the human brain and spinal cord. Examples of neurodegenerative diseases are Alzheimer's disease, Parkinson's disease, Lewy Body Dementia, Huntington's disease, amyotrophic lateral sclerosis, multiple sclerosis, Creutzfeldt-Jakob disease, Friedreich ataxia, motor neuron disease, Batten disease, tauopathies, prion, spinal muscular atrophy and spinocerebellar ataxia. Two major neurodegenerative diseases are Alzheimer's and Parkinson's diseases. Some neurodegenerative disorders are caused by inherited genetic changes. Majority of neurodegenerative disorders are due to a combination of genetic and environmental factors. This makes it difficult to predict who will develop the disease. It is estimated that 55 million people worldwide had dementia in 2019, and that by 2050 this figure will increase to 139 million people.

There is no good way to reverse the neuron degeneration, Oxidative stress and inflammation. are the two major contributors to neural degeneration. Recent research reveals many similarities between these diseases are the build ups of atypical protein assemblies which induced cell death.

Alzheimer's disease (AD) is the most prevalent neurodegenerative disease that usually starts slowly and progressively worsens. As the disease advances, symptoms can include problems with language, disorientation (including easily getting lost), mood swings, loss of motivation, self-neglect, and behavioral issues. Alzheimer's disease is believed to occur when abnormal amounts of amyloid beta (Aβ), accumulating extracellularly as amyloid plaques and tau proteins, accumulating intracellularly as neurofibrillary tangles, form in the brain, affecting neuronal functioning and connectivity, resulting in a progressive loss of brain function. Currently, no treatments stop or reverse its progression, though some may temporarily improve symptoms.

Parkinson's disease (PD) is a long-term degenerative disorder of the central nervous system that mainly affects the motor system. It is sometimes referred to as a type of neurodegenerative disease called synucleinopathy due to an abnormal accumulation of the protein alpha-synuclein in the brain. The most obvious early symptoms of PD are tremor, rigidity, slowness of movement, and difficulty with walking. Cognitive and behavioral problems may also occur with depression, anxiety, and apathy occurring in many people with PD. Dementia becomes common in the advanced stages of PD. No cure for PD is known; treatment aims to reduce the effects of the symptoms.

Lewy body dementia (LBD) is the second most common type of dementia after Alzheimer's disease. The exact cause is unknown but involves abnormal protein deposits developing in the brain and causing neuron cell damage. The abnormal protein aggregation is called Lewy body, the primary structural component of which is alpha-synuclein. Lewy bodies may also be found in other types of dementia, including Alzheimer's disease, multiple system atrophy and Parkinson's disease. LBD is a type of progressive dementia that leads to a decline in thinking, reasoning and problems with movement, behavior, and mood. The symptoms typically begin at age over 50. The progression of the symptoms varies depending on the person's overall health. People with LBD live on average five to eight years and there is current no cure for LBD.

Huntington's disease (HD) is an inherited disorder that causes a protein called huntingtin damaging neuron in the brain. HD causes deterioration in a person's physical, mental, and emotional abilities, Symptoms typically appear in middle age and currently have no cure. People living with HD develop uncontrollable dance-like movements (chorea) and abnormal body postures, as well as problems with behavior, emotion, thinking, and personality. The cognitive changes get worse as the disease progresses, until people with HD are not able to work, drive, or care for themselves.

Anti-CD3 antibody targets the surface receptor on T cells and downregulates inflammation reaction in disease onset. It has been demonstrated that anti-CD3 can regulate T cells and facilitate microglia polarization from M1 to M2 status. M1 microglia secrete inflammatory mediators, triggering inflammation and neurotoxicity, whereas M2 microglia secrete anti-inflammatory mediators, promoting anti-inflammatory responses and neuroprotection.

Clinically, the treatment of anti-CD3 antibody is associated with a wide spectrum of toxic side effects which occur almost immediately after administration of only the first dose. This response is called the cytokine release syndrome (CRS) and occurs in many patients receiving anti-CD3 antibody therapy. CRS is a serious adverse effect due to life threatening systemic inflammatory response in patients. Long term treatment of anti-CD3 will lead to leukopenia (low white blood cell count), which leads to an increased risk of infection. These are current clinical limitations in using anti-CD3 for neurodegenerative diseases and other autoimmune diseases.

The blood-brain-barrier (BBB) is a highly selective semipermeable border of endothelial cells of the central nervous system (CNS) that prevents solutes in the circulating blood vessel from non-selectively crossing into central nervous system where neurons reside. Therefore, BBB is a barrier hindering many effective drugs from entering the brain to treat brain diseases.

There are 4 pathways for BBB penetration, passive diffusion, carrier-mediated transport, receptor-mediated transcytosis, and adsorptive-mediated transcytosis (AMT). AMT pathway uses Caveolae as transporting vehicle. Caveolae-mediated endocytosis is a critical transporting mechanism for macromolecule uptake from blood stream to CNS.

Flavonoids have the general structure of a 15-carbon skeleton, which consists of two phenyl rings (A and B) and a heterocyclic ring (C, the ring containing the embedded oxygen).

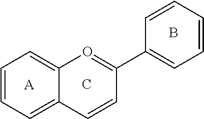

This carbon structure can be abbreviated C6-C3-C6. According to the IUPAC nomenclature, flavonoids can be classified into:
flavonoids or bioflavonoids
isoflavonoids, derived from 3-phenylchromen-4-one (3-phenyl-1,4-benzopyrone) structure
neoflavonoids, derived from 4-phenylcoumarine (4-phenyl-1,2-benzopyrone) structure

### BRIEF DESCRIPTION OF THE DRAWINGS

FIG. 1 illustrates one embodiment of a MINC (Multi-pathway Immune-modulating Nanocomplex Combination therapy)-agent, which is a nanocomplex having a polymer-flavonoid conjugate, for example, a PEG-EGCG conjugate, in a shell and having an agent encapsulated.

FIG. 2 illustrates another embodiment of a MINC-agent, which is a nanocomplex comprises a polymer-flavonoid conjugate, e.g., a PEG-EGCG conjugate in an outer shell and a flavonoid oligomer, for example, oligomeric EGCG (OEGCG), in an inner shell, and having an agent encapsulated.

FIG. 3 shows the nanoparticle size distribution of a nanocomplex composition of the MINC-anti-CD3.

FIG. 4 shows that MINC-Dox penetrated BBB better than Dox.

FIG. 5 shows that MINC-anti-CD3 was protective to neuron cells and inhibited the Aβ-induced cell death.

FIG. 6 shows that OEGCG, PEG-GCGC, and MINC-anti-CD3 promoted neuron cell proliferation.

FIG. 7 shows that MINC-anti-CD3 reduced the PLS stimulated IL-6 secretion from microglia.

FIG. 8 shows that MINC-anti-CD3 decreased the toxicity of anti-CD3, in a body weight experiment and a survival experiment.

FIG. 9 shows that MINC-anti-CD3 treatment was safe in animals and did not alter the blood cell composition compared with saline treated control group.

### DETAILED DESCRIPTION OF THE INVENTION

### Definitions

The term “about” is defined as +10%, preferably +5%, of the recited value.

The term “cytokines” refer to small proteins (<80 kDa) important in cell signaling. Cytokines have been shown to be involved in autocrine, paracrine, and endocrine signaling as immunomodulating agents. Cytokines include interferons, interleukins, lymphokines, tumor necrosis factors, and chemokines.

The term “epigallocatechin gallate” refers to an ester of epigallocatechin and gallic acid, and is used interchangeably with “epigallocatechin-3-gallate” or EGCG.

The term “nanocomplex” refers to a complex in a nanometer size (1-999 nm) that comprises several different components joined together by non-covalent binding such as ionic interaction, hydrophobic interaction, and hydrogen bond.

The term “oligomeric EGCG” (OEGCG) refers to 3-20 monomers of EGCG that are covalently linked. OEGCG preferably contains 4 to 12 monomers of EGCG.

The term “polyethylene glycol-epigallocatechin gallate conjugate” or “PEG-EGCG refers to polyethylene glycol (PEG) conjugated to one or two molecules of EGCG. The term “PEG-EGCG” refer to both PEG-mEGCG conjugate (monomeric EGCG) and PEG-dEGCG (dimeric EGCG) conjugate.

The present disclosure provides a method of treating neurodegenerative diseases.

The method comprises the step of administering to a subject in need thereof an effective amount of (i) a polymer-flavonoid conjugate, (ii) a flavonoid oligomer, or (iii) a nanocomplex having (a) an outer shell comprising one or more polymer-flavonoid conjugates, (b) optionally an inner shell comprising one or more flavonoid oligomer, and (c) an antibody encapsulated within the shells. Anti-CD3 is a preferred antibody drug in the present method. Preferred antibodies for the present method include anti-CD3 and anti-CD33 antibodies.

Free anti-CD3 antibody is known for causing life threatening adverse side effect such as cytokine storm, a fatal aberrant release of cytokines in patients. The present nanocomplex reduces the toxicity anti-CD3 and is effective in treating a neurodegenerative disease such as AD, PD, HD, and LBD.

### Flavonoids

Flavonoids suitable for the present invention have the general structure of Formula I:

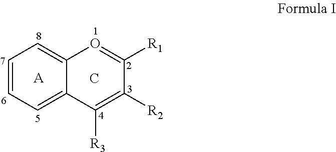

wherein:
R₁ is H, or phenyl;
R₂ is H, OH, Gallate, or phenyl; wherein the phenyl is optionally substituted by one or more (e.g., 2-3) hydroxyl;
R₃ is H, OH, or ═O (oxo); or
R₁ and R₂ together form a close-looped ring structure; or
R₂ and R₃ together form close-looped ring structure.

The 2, 3, 4, 5, 6, 7, or 8 position of Formula I, can be linked to a group containing hydrocarbon, halogen, oxygen, nitrogen, sulfur, phosphorus, boron or metals.

Examples of flavonoids of Formula I include:

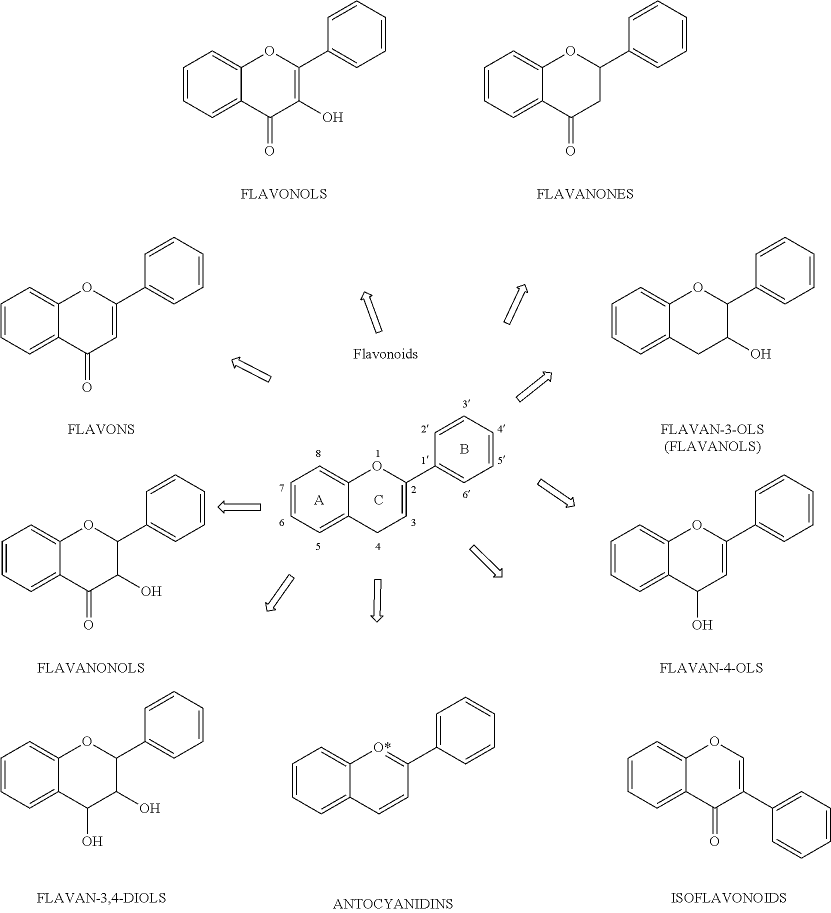

Preferred flavonoid compounds of Formula I include:
EGCG (CAS #989-51-5), EC (CAS #490-46-0), EGC (CAS #970-74-1) or ECG (CAS #1257-08-5)

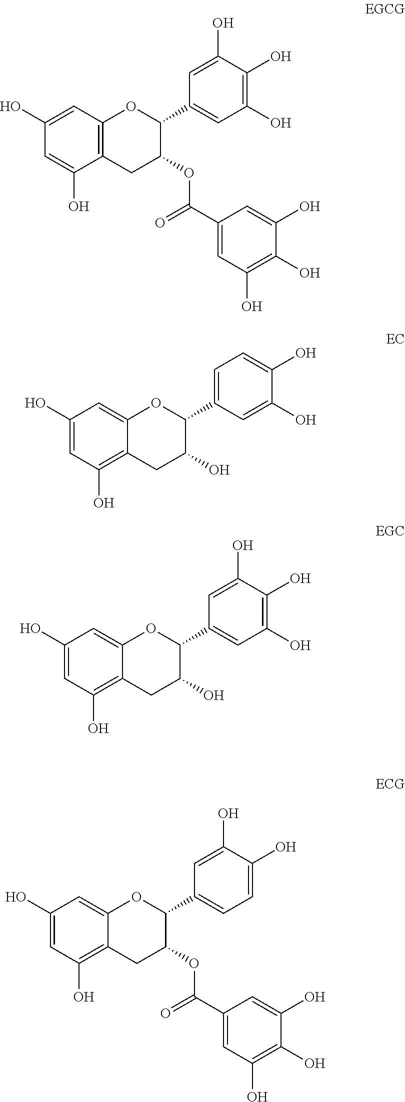 [SMILES: O=C(O[C@@H]1Cc2c(O)cc(O)cc2O[C@@H]1c1cc(O)c(O)c(O)c1)c1cc(O)c(O)c(O)c1 ; Oc1cc(O)c2c(c1)O[C@H](c1ccc(O)c(O)c1)[C@H](O)C2 ; Oc1cc(O)c2c(c1)O[C@H](c1cc(O)c(O)c(O)c1)[C@H](O)C2 ; O=C(O[C@@H]1Cc2c(O)cc(O)cc2O[C@@H]1c1ccc(O)c(O)c1)c1cc(O)c(O)c(O)c1]

### Polymer-Flavonoid Conjugate

A polymer-flavonoid conjugate, as used herein throughout the application, refers to a conjugate of a hydrophilic polymer and the flavonoid compound of Formula I.

A hydrophilic polymer refers to a polymer that is soluble in polar solvents and can form hydrogen bonds. Hydrophilic polymers suitable for the present polymer-flavonoid conjugates include, but not limited to: poly(ethylene glycol), aldehyde-derivatized hyaluronic acid, hyaluronic acid, dextran, diethylacetal conjugate (e.g. diethylacetal PEG), D-alpha-tocopheryl polyethylene glycol succinate, aldehyde-derivatized hyaluronic acid-tyramine, hyaluronic acid-aminoacetylaldehyde diethylacetal conjugate-tyramine, cyclotriphosphazene core phenoxymethyl(methylhydrazono)dendrimer or thiophosphoryl core phenoxymethyl(methylhydrazono)dendrimer. acrylamides, oxazolines, imines, acrylic acids, methacrylates, diols, oxiranes, alcohols, amines, anhydrides, esters, lactones, terephthalate, amides and ethers polyacrylamide, poloxamers, poly(N-isopropylacrylamide), poly(oxazoline), polyethylenimine, poly(acrylic acid), polymethacrylate, poly(ethylene glycol), poly(ethylene oxide), poly(vinylalcohol), poly(vinylpyrrolidinone), polyethers, poly(allylamine), polyanhydrides, poly(β-amino ester), poly(butylene succinate), polycaprolactone, polycarbonate, polydioxanone, poly(glycerol), polyglycolic acid, poly(3-hydroxypropionic acid), poly(2-hydroxyethyl methacrylate), poly(N-(2 hydroxypropyl)methacrylamide), polylactic acid, poly(lactic-co-glycolic acid), poly(ortho esters), poly(2 oxazoline), poly(sebacic acid), poly(terephthalate-co-phosphate), povidone and copolymers.

Preferred hydrophilic polymers include poly(ethylene glycol), hyaluronic acid, dextran, polyethylenimine, poloxamers, povidone, D-alpha-tocopheryl and polyethylene glycol succinate. The molecular weight of the hydrophilic polymer in the polymer-flavonoid conjugate is in general 1K-100K Daltons, preferably 2K-40K, 2K-50K, 2K-80K, 3K-80K, or 5K-40K Daltons.

In one embodiment, the polymer contains an aldehyde group which is conjugated to the 5, 6, 7, or 8 position (preferably 6 or 8 position) of the A ring of the flavonoid compound. In another embodiment, the polymer contains a thiol group which is conjugated to R₁ or R₂ of the B-ring of a flavonoid (when R₁ or R₂ is —OH).

In one embodiment, the polymer-flavonoid conjugate is PEG-EGCG, which is PEG conjugated to one or two molecules of epigallocatechin gallate (EGCG). PEG-EGCG, for example, can be prepared by conjugating aldehyde-terminated PEG to EGCG by attachment of the PEG via reaction of the free aldehyde group with the 5, 6, 7, or 8 position (preferably 6 or 8 position) of Formula I. See WO2006/124000 and WO2009/054813. PEG-EGCG can also be prepared by conjugating thio-terminated PEG to EGCG by attachment of the PEG via reaction of the free thio group with the R1 or R2 of Formula I, wherein, R1 or R2 is a phenyl group. See WO2015/171079.

### Flavonoid Oligomers

A flavonoid oligomer is a conjugate of one flavonoid with one or more flavonoids. The flavonoid oligomer can contain the same flavonoid (a homo oligomer) or different flavonoids (a hetero oligomer). Flavonoid oligomers useful for the present invention in general have 2-50 or 2-20, preferably 4-12 flavonoids of one or mixed types.

In some embodiment, a flavonoid oligomer is oligomeric EGC (OEGCG), oligomer EC (OEC), oligomer EGC (OEGC), or oligomer ECG (OECG). OEGCG refers to 3-20 monomers of EGCG that are covalently linked. OEGCG, for example, can be synthesized at 5, 6, 7, or 8 position (preferably 6 or 8 position) of the A ring according to WO2006/124000.

Because A-ring is present in all of the flavonoids according to Formula 1, other oligomeric flavonoids can be made similarly according to WO2006/124000. For example, OEC, OEGC, and OECG can also be made according to WO2006/124000.

### MINC-Agent

MINC (Multi-pathway Immune-modulating Nanocomplex Combination therapy) is a platform technology, utilizing the bioactivity of polymer-flavonoid conjugates or flavonoid oligomers that form a nanocomplex in a solution.

MINC platform can encapsulate agents to form a nanoparticle composition for a therapy. MINC-agent is a nanocomplex having an outer shell comprises one or more polymer-flavonoid conjugates and optionally an inner shell comprises one or more flavonoid oligomer and a drug within the shells. The agent, as used herein, reference to a molecule that have a therapeutic activity (e.g., a drug).

In one embodiment, the MINC-agent is in a form of micelle.

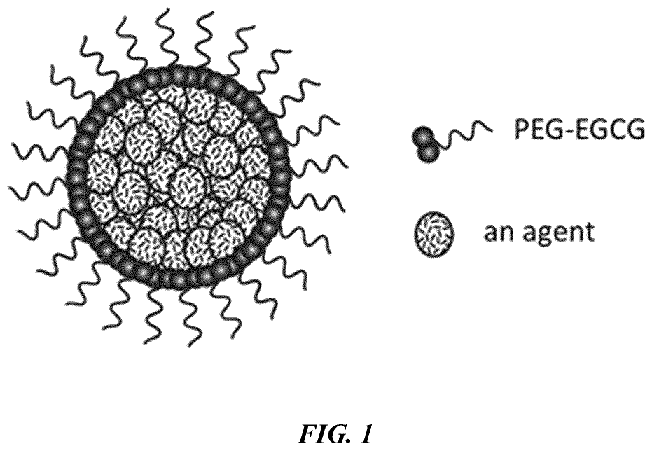

In one embodiment, MINC-agent is a nanocomplex comprises a polymer-flavonoid conjugate, for example, a PEG-EGCG conjugate, in a shell and an agent encapsulated within the shell (see FIG. 1).

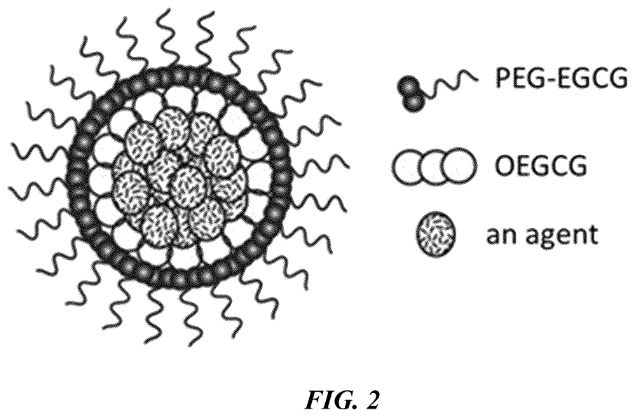

In another embodiment, MINC-agent is a nanocomplex comprises a polymer-flavonoid conjugate, for example, a PEG-EGCG conjugate in an outer shell and a flavonoid oligomer, for example, oligomeric EGCG (OEGCG), in an inner shell, with an agent encapsulated within the cells encapsulated (see FIG. 2).

When the agent is a drug, the MINC-Agent composition comprises two or more components that have therapeutic activities, which are complementary in function to form a multiple targeted combination therapy by its backbone components (a flavonoid conjugate or a flavonoid oligomer), and the encapsulated agent.

In one embodiment, the agent in MINC-agent is an antibody including but not limited to anti-CD3, anti-CD39, anti-CD73, anti-PD-L2, anti-CTLA-4, anti-GZM A, anti-GZM-B, anti-CD33, anti-TAM, anti-FcγRI, anti-CD36, anti-RAGE, anti-APOE, or anti-CR1.

For example, the MINC-agent is anti-CD3, the polymer-flavonoid conjugate is PEG-EGCG, and the flavonoid oligomer is OEGCG. This is one preferred composition for treating a neurodegenerative disease, such as Alzheimer's disease, Parkinson's disease, Lewy body dementia, and Huntington's disease.

### Pharmaceutical Composition

The present invention provides pharmaceutical compositions comprising the polymer-flavonoid conjugate, flavonoid oligomer, or MINC-agent as described in the application, and optionally one or more pharmaceutically acceptable excipients. The nanoparticle component in a pharmaceutical composition in general is about 1-100% or 1-90%, preferably 20-90%, or 30-80% for a tablet, powder, or parenteral formulation. The polymer-flavonoid conjugate, flavonoid oligomer, or MINC-agent composition in a pharmaceutical composition in general is 1-100%, preferably 20-100%, 50-100%, or 70-100% for a capsule formulation. The nanoparticle composition in a pharmaceutical composition in general is 1-50%, 5-50%, or 10-40% for a liquid suspension formulation.

In one embodiment, the pharmaceutical composition can be in a dosage form such as tablets, capsules, granules, fine granules, powders, suspension, solution, patch, parenteral, injectable, or the like. The above pharmaceutical compositions can be prepared by conventional methods.

Pharmaceutically acceptable carriers, which are inactive ingredients, can be selected by those skilled in the art using conventional criteria. The pharmaceutically acceptable carriers may contain ingredients that include, but are not limited to, saline and aqueous electrolyte solutions; ionic and nonionic osmotic agents, such as sodium chloride, potassium chloride, glycerol, and dextrose; pH adjusters and buffers, such as salts of hydroxide, phosphate, citrate, acetate, borate, and trolamine; antioxidants, such as salts, acids, and/or bases of bisulfite, sulfite, metabisulfite, thiosulfite, ascorbic acid, acetyl cysteine, cysteine, glutathione, butylated hydroxyanisole, butylated hydroxytoluene, tocopherols, and ascorbyl palmitate; surfactants, such as lecithin and phospholipids, including, but not limited to, phosphatidylcholine, phosphatidylethanolamine and phosphatidyl inositol; poloxamers and poloxamines; polysorbates, such as polysorbate 80, polysorbate 60, and polysorbate 20; polyethers, such as polyethylene glycols and polypropylene glycols; polyvinyls, such as polyvinyl alcohol and polyvinylpyrrolidone (PVP, povidone); cellulose derivatives, such as methylcellulose, hydroxypropyl cellulose, hydroxyethyl cellulose, carboxymethyl cellulose, and hydroxypropyl methylcellulose and their salts; petroleum derivatives, such as mineral oil and white petrolatum; fats, such as lanolin, peanut oil, palm oil, and soybean oil; mono-, di-, and triglycerides; polysaccharides, such as dextrans; and glycosaminoglycans, such as sodium hyaluronate. Such pharmaceutically acceptable carriers may be preserved against bacterial contamination using well-known preservatives, which include, but are not limited to, benzalkonium chloride, ethylene diamine tetra-acetic acid and its salts, benzethonium chloride, chlorhexidine, chlorobutanol, methylparaben, thimerosal, and phenylethyl alcohol, or may be formulated as a non-preserved formulation for either single or multiple use.

For example, a tablet, capsule, or parenteral formulation of the active compound may contain other excipients that have no bioactivity and no reaction with the active compound. Excipients of a tablet or a capsule may include fillers, binders, lubricants and glidants, disintegrators, wetting agents, and release rate modifiers. Examples of excipients of a tablet or a capsule include, but are not limited to, carboxymethylcellulose, cellulose, ethylcellulose, hydroxypropylmethylcellulose, methylcellulose, karaya gum, starch, tragacanth gum, gelatin, magnesium stearate, titanium dioxide, poly(acrylic acid), and polyvinylpyrrolidone.

For example, a tablet formulation may contain inactive ingredients, such as colloidal silicon dioxide, crospovidone, hypromellose, magnesium stearate, microcrystalline cellulose, polyethylene glycol, sodium starch glycolate, and titanium dioxide. A capsule formulation may contain inactive ingredients, such as gelatin, magnesium stearate, and titanium dioxide. A powder oral formulation may contain inactive ingredients, such as silica gel, sodium benzoate, sodium citrate, sucrose, and xanthan gum.

The pharmaceutical composition can be applied by local administration and systemic administration. Local administration includes topical administration. Systemic administration includes oral, parenteral (such as intravenous, intramuscular, subcutaneous, or rectal), and other systemic routes of administration. In systemic administration, the active compound first reaches plasma and then distributes into target tissues. Parenteral administration, such as intravenous bolus injection or intravenous infusion, and oral administration are preferred routes of administration.

### Method of Treatment

The present invention is directed to a method of treating or preventing relapse of neurodegenerative diseases, by administering to a subject in need thereof a polymer-flavonoid conjugate, flavonoid oligomer, or MINC-agent, as described above.

Suitable neurodegenerative diseases to be treated by the present invention include, but not limited to Alzheimer's disease (AD), Parkinson's disease (PD), Lewy Body Dementia (LBD), Huntington's disease (HD), amyotrophic lateral sclerosis (ALS), multiple sclerosis (MS), Creutzfeldt-Jakob disease (CJD), Friedreich ataxia (FA), motor neuron disease (MND), Batten disease, spinal muscular atrophy (SMA) and spinocerebellar ataxia (SCA). The present method is particularly useful in treating AD, PD, LBD, and HD.

### Polymer-Flavonoid Conjugate, Flavonoid Oligomer

The present invention is directed to a method for treating a neurogenic disease by administering one or more polymer-flavonoid conjugate and/or one or more flavonoid oligomer.

The method comprises the step of administering to a subject in need thereof an effective amount of one or more polymer-flavonoid conjugates and/or one or more flavonoid oligomers, to treat a neurodegenerative disease. The polymer-flavonoid conjugates and the one or more flavonoid oligomers are described above in this application.

EGCG is capable to reduce misfolded β-amyloid and tau protein aggregation for Alzheimer's disease, α-synuclein for Parkinson's disease and Lewy body dementia, and Huntingtin for Huntington's disease. EGCG also exerts neuron protective function in these neurodegenerative diseases. Mechanically, EGCG has neuron protective functions on neuron cells directly. EGCG has antioxidant activity by working as free radical scavenger and anti-apoptotic activity by reducing expression of proapoptotic genes. The EGCG in MINC-agent provides benefits of protecting neural cells from damage by toxins. EGCG is also known to promote neuron cell proliferation and regeneration.

In one embodiment, the polymer is a hydrophilic polymer having a molecular weight of 1,000 to 100,000 Daltons, and is selected from the group consisting of: poly(ethylene glycol) (PEG), hyaluronic acid, dextran, polyethylenimine, poloxamers, povidone, D-alpha-tocopheryl, and polyethylene glycol succinate;

In one embodiment, the flavonoid oligomer comprises 2-50 flavonoids of EGCG, EC, EGC, or ECG.

In one embodiment, the shell is formed by PEG-EGCG.

In one embodiment, the shell is formed by PEG-EGCG and OEGCG.

One important function of polymer-flavonoid conjugate and flavonoid oligomer is to increase drug delivery to the brain (crossing BBB) to enhance therapeutic efficacy. This function is due to the ability of polymer-flavonoid conjugate to penetrate BBB. Drug molecules are encapsulated, and they are not exposed to the BBB and thus they have no influence on entering CNS. This brain delivery applies to neurodegenerative diseases (Alzheimer's disease, Parkinson's disease, Lewy Body Dementia, Huntington's disease, amyotrophic lateral sclerosis, multiple sclerosis, Creutzfeldt-Jakob disease, Friedreich ataxia, motor neuron disease, Batten disease, spinal muscular atrophy and spinocerebellar ataxia.).

Another function of polymer-flavonoid conjugate and flavonoid oligomer is to reduce neural cell death and enhance cell regeneration to restore cognitive behavior. This function treats neurodegenerative diseases such as Alzheimer's disease, Parkinson's disease, Lewy body dementia, and Huntington's disease.

Another function of polymer-flavonoid conjugate and flavonoid oligomer is to reduce neural cell oxidative stress and inflammation caused by the abnormal protein aggregates. Oxidative stress leads to neural toxicity and is involved in the development of neurodegenerative diseases. Reducing neural cell oxidative stress reduces neuronal cell death and treats neurodegenerative diseases (Alzheimer's disease, Parkinson's disease, Lewy body dementia, Huntington's disease).

Another function of polymer-flavonoid conjugate and flavonoid oligomer is to reduce accumulation of abnormal proteins (e.g., β-amyloid, Tau, and α-synuclein) and to delay and/or reduce the progression and/or recurrence of diseases. These processes treat neurodegenerative diseases (Alzheimer's disease, Parkinson's disease, Lewy body dementia, and Huntington's disease).

### MINC-Agent

The present invention is directed to a method for treating a neurogenic disease by administering a MINC-Agent. The method comprises the step of administering to a subject in need thereof an effective amount of a nanocomplex comprising: (a) an outer shell comprising one or more polymer-flavonoid conjugates, (b) optionally an inner shell comprising one or more flavonoid oligomer, and (c) anti-CD3 antibody or anti-CD33 antibody encapsulated within the shells, to treat a neurodegenerative disease.

In one embodiment, the outer shell is formed by one or more polymer-flavonoid conjugates. In one embodiment, the inner shell is formed by one or more flavonoid oligomer. In one embodiment, the agent is an antibody.

The neurodegenerative disease suitable to be treated by the present method is Alzheimer's disease, Parkinson's disease, Lewy Body Dementia, Huntington's disease, amyotrophic lateral sclerosis, multiple sclerosis, Creutzfeldt-Jakob disease, Friedreich ataxia, motor neuron disease, Batten disease, spinal muscular atrophy and spinocerebellar ataxia.

As already described above, a polymer-flavonoid conjugate or a flavonoid oligomer is capable of crossing BBB from the circulating blood vessel to the brain as an agent delivery vehicle to deliver said agent for treating neurodegenerative disorders.

Other functions of a polymer-flavonoid conjugate or a flavonoid oligomer have already been described above.

In one embodiment, the agents (or drugs) in MINC-agent can modulate immune T cell and microglia activity. The agents include but not limit to the antibodies below. Preferred antibodies include anti-CD3 antibody.

Anti-CD3 antibody can regulate T cells and microglia to reduce neural inflammation and are effective in treating Alzheimer's disease (AD). Anti-CD3 meliorates AD by protecting neuron from damage caused by Aβ accumulation, and polarizing brain microglia's phenotype from M1 (causing inflammation-damaging type) to M2 (plaque clearing-protective type).

Anti-CD3 antibody is useful for treating Parkinson's disease (PD) by decrease the totally CD3⁺ T cell number in blood. PD disease severity is significantly correlated with the observed decrease of CD3⁺ T cells. The improvement of PD symptoms including reducing tremor, rigidity, nerve pain, and/or dystonia.

Huntington disease (HD) is an inherited neurodegenerative disease with abnormal Huntingtin protein accumulation in the brain. It is suitable for early drug intervention. Several preclinical and clinical trials of potential immunomodulatory drugs (including anti-CD3) have been investigated in HD. The abnormal inflammatory situation in central nervous system and neuron damage in HD are similar to other neurodegenerative diseases such as Alzheimer's disease and Parkinson's disease. Several preclinical and clinical trials of potential immunomodulatory drugs (including anti-CD3) have been investigated in HD. Suppressed markers of immune activation in blood and improved cortical motor activity was observed.

However, free anti-CD3 is known for causing life threatening adverse side effect such as cytokine storm, a fatal aberrant release of cytokines in patients. The present MINC-anti-CD3 reduces the toxicity of anti-CD3 and is effective in treating AD, PD, HD, and LBD.

Anti-CD33 inhibits the high CD33 surface expression that impairs the phagocytic capacity of microglia and contributes to the risk for Alzheimer's disease.

Anti-CD39 antibody modulates ectonucleotidases to break down ATP, which can inhibit proinflammatory cytokine production.

Anti-CD73 antibody modulates ectonucleotidases to break down ATP, which can inhibit proinflammatory cytokine production.

Anti-PD-1 serves as systemic PD-1 blockade promotes clearance of cerebral amyloid-β plaques and improves memory in Alzheimer's disease (AD).

Anti-PD-L1 elevates levels of effector memory T cells in the periphery, which is followed by an increased number of monocyte-derived macrophages in the brain parenchyma and secret IL-10, inhibitory cytokine to inflammation.

Anti-PD-L2 inhibits T-cell proliferation by blocking cell cycle progression and it decreases the brain infiltration of macrophage and B cell during multiple sclerosis and Alzheimer's disease progression.

Anti-CTLA-4 activates immune tolerance on T cells; the stimulation is beneficial in autoimmune disease, like AD and MS.

Anti-GZM A/B inhibits Granzyme B, which is released by activated T cells and is a cytotoxic proteinase associated with acute and subacute brain inflammatory disorders in neurogenerative disease.

Interleukin-2 improves amyloid pathology, synaptic failure and memory in Alzheimer's disease.

Anti-TREM2 induces activation of microglia and improves cognitive function in Alzheimer's disease.

Anti-TAM serves as an agonist to promote microglial phagocytosis of apoptotic cells to maintain brain homeostasis.

Anti-scavenger receptors modulate microglia to induce phagocytosis of Abeta.

Anti-FcγR inhibits FcγR induced pro-inflammatory response including the release of cytokines and other mediators in microglia during AD progression.

Anti-CD36 serves as inhibitors of CD36-Amyloid Beta binding to avoid the activation of microglia for AD progression.

Anti-RAGE interrupts the RAGE-Abeta interaction and downstream of ROS production in microglia, astrocyte and endothelial during AD progression.

Anti-APOE can inhibit plaque-induced microglial reactivity and lipid metabolism to promote inflammation.

Anti-CR1 modulates microglia in immune clearance of Aβ in AD brain.

Anti-CD38 controls multi-function of microglia, NAD metabolism and proinflammatory cytokines production, to reduce neuroinflammation under AD progression.

In one embodiment, the neurodegenerative disease is Alzheimer's disease, Parkinson's disease, Huntington's disease or Lewis body dementia, and the drug is anti-CD3, anti-CD39, anti-CD73, anti-PD-L2, anti-CTLA-4, anti-GZM A, anti-GZM-B, anti-CD33, anti-TAM, anti-FcγRI, anti-CD36, anti-RAGE, anti-APOE, anti-CR1 and anti-CD38.

Dosing of the MINC-agent is based on the known dosage of the agents for treating a particular disease and the subject condition. The dosage can be a food drug administration (FDA) approved dosage or a dosage used in clinical trial.

In MINC-agent, in general, the dosage of PEG-EGCG combined with OEGCG is between 10 μg/kg to 10 mg/kg.

The concentration for the drug agents in MINC can be as low as 10 μg/kg and as high as 10 mg/kg.

For treating Alzheimer disease, MINC-anti-CD3 can be administered at a range of 0.01-10 mg anti-CD3/kg, IV once every one to four weeks. After MINC-anti-CD3 treatment, the oxidative stress in the brain is reduced, and regeneration of the neuron is stimulated. This helps relieve disease symptoms such as difficulties in communicating, judgment, understanding, remembering, and moving. To prevent the relapse of the symptoms, repeat dosing of 0.005-5 mg every three to six months would prove suitable. The concentrations of biomarkers such as Aβ42, Aβ40, t-Tau, p-Tau, and proinflammatory cytokines in peripheral circulation in conjunction with brain imaging are monitored.

For treating Parkinson's disease, MINC-anti-CD3 can be administered at a range of 0.01-10 mg anti-CD3/kg, IV once every one to four weeks. MINC-anti-CD3 treatment inhibits the abnormal protein accumulation in the brain area which is in charges of mobility. Improvement of symptoms such as rhythmic shaking (tremor), slowed movement (bradykinesia) and rigid muscles is observed after the MINC-anti-CD3 treatment. To prevent the relapse of the symptoms, repeat dosing of 0.005-5 mg every three to six months would prove suitable. The concentrations of biomarkers (such as alpha-synuclein, proinflammatory cytokines) in peripheral circulation in conjunction with brain imaging will be monitored.

For treating Huntington's disease, MINC-anti-CD3 can be administered at a range of 0.01-10 mg anti-CD3/kg, IV once every one to four weeks. MINC-anti-CD3 inhibits the regional formation of huntingtin plaque, and reduces the oxidative stress in patient's brain. MINC-anti-CD3 slows the progression of the disease by reducing major symptoms include depression, mood swings and personality changes, stumbling and clumsiness, involuntary jerking or fidgety movements of the limbs. To prevent the relapse of the symptoms, repeat dosing of 0.005-5 mg every three to six months would prove suitable. The concentrations of biomarkers (such as mHtt, proinflammatory cytokines) in peripheral circulation in conjunction with brain imaging are monitored.

For treating Lewy body dementia, MINC-anti-CD3 can be administered at a range of 0.01-10 mg anti-CD3/kg, IV once every one to four weeks. The aggregation of Lewy body in patients' brain is reduced by MINC-anti-CD3. A patient recovers from major disease symptoms such as visual hallucinations, fluctuating cognition, sleep behavior disorder and spontaneous changes in attention. To prevent the relapse of the symptoms, repeat dosing of 0.005-5 mg every three to six months would prove suitable. The concentrations of biomarkers (such as proinflammatory cytokines) in peripheral circulation in conjunction with brain imaging are monitored.

Anti-CD3 antibody binds to the CD3 receptor on T cells and downregulating inflammation, decreasing IL-1β and TNFα secretion from microglia, polarizing microglia from M1 (damaging) to M2 (protective) phenotype.

Anti-CD3 monoclonal antibody alone has been applied to the treatment of type I diabetes and Alzheimer's disease. But the severe clinical toxicity has hindered its further clinical development. When Anti-CD3 antibody is encapsulated with OE/PE, the immune modulating function of the antibody is preserved, and the whole MINC-antiCD3 complex is no longer toxic.

Clinically, anti-CD3 monoclonal antibody has been approved for treating T1D but due to drug toxicity, the current therapeutic course is limited to IV injection at 1 to 20 μg/kg for a 14-day course, and only once for everyone's lifetime without repeat dosing. For T1D and neurodegenerative diseases (AD, PD, HD and LBD), MINC-anti-CD3 in the same dose range can be administered with repeating dose for long term cognition rejuvenation since there is no toxicity issue.

The present invention is useful in treating human and non-human animals. For example, the present invention is useful in treating subjects, such as humans, horses, pigs, cats, dogs, or rodents.

The following examples further illustrate the present invention. These examples are intended merely to be illustrative of the present invention and are not to be construed as being limiting.

### EXAMPLES

### Active Ingredients (For All Examples)

### OEGCG:

OEGCG is oligomerized EGCG. OEGCG is prepared according to WO2009/054813.

### PEG-EGCG:

PEG-EGCG is PEG conjugated with one or two EGCG. PEG-EGCG is prepared according to WO2009/054813.

### MINC-Agents:

MINC-agents are made according to WO2009/054813. Alternatively, MINC-agents can be prepared by encapsulated an agent within the nanocomplex formed by PEG-EGCG and OEGCG, according to the method in WO2009/054813.

### Example 1: Method for Preparing MINC-Anti-CD3

### Materials

Anti-CD3 was purchased from Biolegend.

MINC-ant-CD3 was made according to WO2009/054813

MINC-anti-CD3 nanoparticles (PEG-EGCG, OEGCG and anti-CD3) were prepared according to WO2011/112156.

### Method

DLS (Anton Paar Litesizer 500) was used to measure the size of MINC-anti-CD3 nanoparticles.

### Results

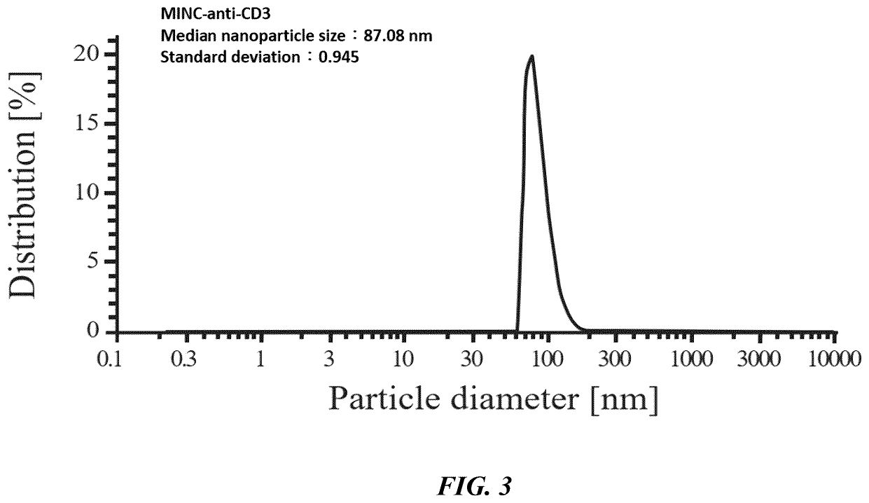

Nanoparticle size was measured by DLS (Anton Paar Litesizer 500). Final particle is shown in FIG. 3 with the median nanoparticle size of 87.08 nm. The standard deviation was 0.945. More than 95% of the nanoparticles were distributed within 50-300 nm.

### Example 2: MINC-Agent Platform Delivers Drugs to Penetrate BBB in Surrogate Cell Model

### Materials

OEGCG, PEG-EGCG, and MINC-Doxorubin are the same as described in Example 1.

### Method

To confirm the efficacy of MINC platform in bringing drugs across blood brain barrier, we selected doxorubicin (a chemotherapy medicine which cannot pass through BBB) for in vitro BBB transwell study.

In brief, 3×10⁴ Caco-2 cells were seeded into the luminal side of the insert of 24 well transwell plate (Falcon) to simulate the BBB. Medium was changed every 3 days. Transepithelial electrical resistance (TEER) value which indicated the BBB barrier integrity of each well was measured by a Millicell ERS Voltohmmeter (Millipore, MA, USA). When the TEER value of each insert reached 250 Ω*cm², either (1) 5 μg/mL unencapsulated free doxorubicin or (2) MINC-doxorubicin with fluorescent intensity equivalent to 5 μg/mL doxorubicin were added to the top of inserts. After 8 hours of incubation, medium in the upper insert and the lower culture well was collected and fluorescence signal (relative fluorescence unit, RFU) at Ex/Em=470/595 nm was detected by Spectramax i3x. Drug penetration percentage was calculated following the formula: penetration (%)=(RFU_{Lower}×7)÷(RFU_{Upper}×7+RFU_{Lower}×7)

### Results

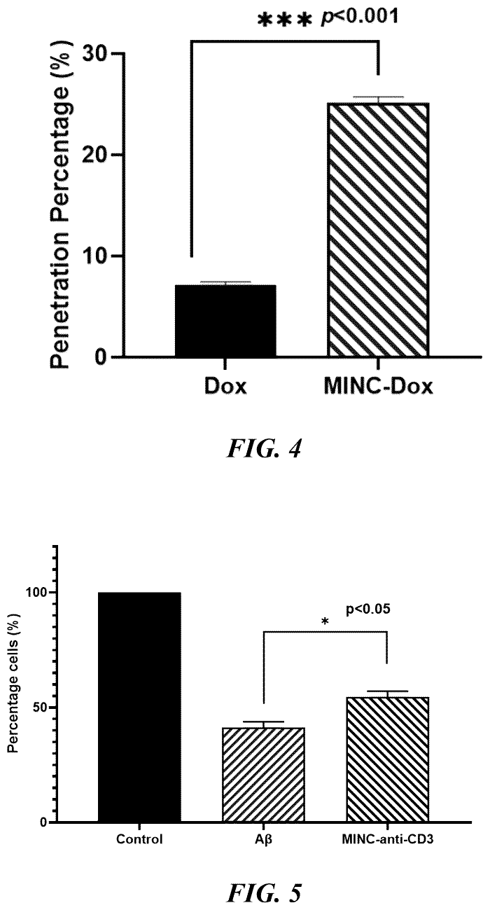

The results are shown in FIG. 4; Significantly more fluorescence signals were observed in MINC-doxorubicin treatment group when compared with doxorubicin alone control group (n=3, p<0.001). The results demonstrate that our MINC-agent platform augmented the BBB penetration ability of doxorubicin in the surrogate BBB transwell model.

### Example 3: MINC-Agent Platform Delivers an Antibody Agent, Anti-HER2, to the Brain in Mouse

### Materials

OEGCG and PEG-EGCG were prepared according to Example 1.

Cyanine5.5 NHS ester (Cy 5.5) (Aladdin) was used.

Anti-HER2-Cy5.5 conjugate was prepared by reacting the anti-HER2 antibody with Cy5.5-NHS ester according to manufacturer's instructions (Aladdin).

MINC-anti-HER2-Cy5.5 is anti-HER2-Cy5.5 encapsulated within PEG-EGCG and OEGCG, and it is prepared according to WO2009/054813.

### Method

Antibody drugs cannot penetrate BBB into brain parenchyma efficiently. Antibody drugs show similar structure, composed with Fab and Fc region, and molecular size (near 150 kDa). In this example, we selected fluorophore labeled anti-HER2 (trastuzumab) as an example to demonstrate that MINC formulation can bring antibody drugs to the brain in mouse model.

Athymic Nude-Foxn1^{nu} female mice at the age of 6 weeks were used, the mice were divided into two groups. One group (n=3) received 10 mg/kg anti-HER2-Cy5.5 through tail vein i.v. bolus as control. The other group (n=3) received equivalent anti-HER2-Cy5.5 dose in MINC-anti-HER2-Cy5.5 via tail vein i.v. bolus. After drug administration, the live images were observed at 8 hour, with ex/em of 674/692 nm, using an IVIS (in vivo imaging system) Lumina III XRMS.

### Results

The results showed that significant high fluorescence signal was present in the brain region of mice receiving MINC-anti-HER2-Cy5.5 (STM-001) but not in the mice treated with anti-HER2-Cy5.5.

Trastuzumab is a FDA approved medicine for HER2+ breast cancer therapy, but it is not approved for glioma because of its poor penetration into the brain. This example shows that MINC-agent platform can deliver antibody agent into the brain in mouse.

The mechanism that MINC-agent platform brings anti-HER2 into the brain applies to other antibodies including anti-CD3.

### Example 4: MINC-Anti-CD3 Inhibited Aβ-Induces Neuron Cell Death

### Materials

Recombinant Aβ (1-42) peptide, MTT (3-(4,5-Dimethylthiazol-2-yl)-2,5-Diphenyltetrazolium Bromide) were purchased from Genscript, Thermo Fisher. HT-22 cell was obtained from Millipore (Bedford, MA, USA)

MINC-anti-CD3 was prepared according to Example 1.

### Method

To confirm the efficacy of MINC-anti-CD3 in protecting Aβ-induced neural cell death, an in vitro MTT assay was conducted. In brief, HT-22 cells were seed 2×10⁵ per well in 24-well dish and maintained in a logarithmic growth phase for 3 days. The cells were then treated with prepared 2.5 μM oligo-Aβ, Aβ+MINC-anti-CD3 for 24 hours. After incubation, cells were washed once with warmed PBS to remove test materials and tetrazolium salt was added for 30 mins at room temperature. Then the formazan product was measured spectrophotometrically at 550 nm. The viability was calculated as percent of untreated control cells.

### Results

FIG. 5 shows that MINC-anti-CD3 is protective to Aβ treated neuron cells. When the neuron cell line was treated with Aβ peptide, the cell viability (% of cell number) decreased to lower than 40% comparing to untreated group. After MINC-anti-CD3 was also added into the Aβ treated cells, MINC-anti-CD3 had protective effect and significantly increased the cell viability to more than 50%.

The accumulation of abnormal protein (such as Aβ or α-synuclein) in the brain is commune in neuron degenerative disease like Alzheimer's disease, Parkinson's disease, Huntington's disease and Lowey body dementia. The data demonstrates that MINC-anti-CD3 protects neuron cells from the damage caused by the accumulation of abnormal proteins.

### Example 5: OEGCG and PEG-EGCG Suppress Aβ-Induced Oxidative Stress

### Materials

Recombinant Aβ (1-42) peptide, DCFH-DA were purchased from Genscript, Thermo Fisher.

HT-22 cell is obtained from Millipore (Bedford, MA, USA)

### Method

This experiment was to test the efficacy of OEGCG, PEG-EGCG and MINC-anti-CD3 in reducing Aβ-induced oxidative stress. In vitro cell reactive oxygen species (ROS) staining test was conducted. HT-22 cells were seeded 2×10⁵ cells per well in 24-well dish and maintained for 3 days. Then cells were then treated with Aβ, Aβ+OEGCG, Aβ+PEG-EGCG and Aβ+MINC-anti-CD3. Oligo-Aβ was prepared. In brief, Aβ peptide was dissolved in 1 mM in 100% 1,1,1,3,3,3-hexafluoro-2-propanol and was dried using a vacuum desiccator. Next, Aβ was resuspended to a concentration of 5 mM in dimethylsulfoxide (DMSO) and stored at −20° C. To obtain oligomers, Aβ peptide was diluted to a final concentration of 100 M by using Dulbecco's modified Eagle's medium (DMEM; Gibco), incubated at 4° C. after 24 h of gentle shaking, and immediately added to cell cultures at a final concentration of 2.5 μM. After cells were treated with Aβ for 1 hour, 10 μm of OEGCG or PEG-EGCG was added to cell and incubate for 6 hours. Then cells were treated with 20 μM of DCFH-DA for 0.5 h at 37° C. under 5% CO₂. After DCFH-DA staining, cells were washed twice with DMEM and once with phosphate-buffered saline to remove background signals. The fluorescent images were collected by fluorescence microscopy (DP72/CKX41, Olympus), and all images were used the same fluorescent conditions and exposure time.

### Results

A fluorescence dye DCFH-DA was used to measure the level of oxidative stress induced by Aβ stimulation. Cells with higher oxidative stress had higher fluorescence intensity. The results demonstrate that the Aβ treated control group yielded most florescent positive cell number (94.4% of total cells) and highest fluorescence intensity of the florescent positive cells; the Aβ+OEGCG (20 M) group had less florescent positive cell number (12.5% of total cells) and the florescence intensity of the positive cells was very low. The Aβ+, PEG-ECGC (20 μM) group had the least florescent positive cell number (11.3% of total cells) and also the florescence intensity of the positive cells was very low.

The oxidative stress is known to cause neuron cell death in various neurodegenerative diseases including Alzheimer's disease, Parkinson's disease, Huntington disease and Lewy body dementia. The data show that MINC ingredients OEGCG and PEG-EGCG and MINC-agent protect neuron cells from oxidative stress-mediated neural damage in these neurodegenerative diseases.

### Example 6: OEGCG, PEG-ECGC and MINC-Anti-CD3 Promote Neuron Cell Proliferation/Regeneration

### Materials

OEGCG and PEG-EGCG were prepared according to Example 1.

MTT (3-(4,5-Dimethylthiazol-2-yl)-2,5-Diphenyltetrazolium Bromide) were purchased from Abcam.

HT-22 cell was obtained from Millipore (Bedford, MA, USA)

MINC-anti-CD3 was prepared according to Example 1.

### Method

We used in vitro cell proliferation assay (MTT assay) to confirm that OE, PE and MINC-anti-CD3 could promote neuro cell proliferation/regeneration. Neuron cells (HT-22) were treated with OE, PE, and MINC-anti-CD3, and an in vitro MTT assay is conducted.

In brief, HT-22 cells were seeded at 2.5×10³ per well in DMEM with 5% FBS in 96-well plate and maintained in a logarithmic growth phase for 24 hours. The cells were then treated with OE (2.2 μg/mL), PE (4 μg/mL) and MINC-anti-CD3 (0.74 μg/mL) for 48 hours. After incubation, cells were washed once with warmed PBS to remove test materials and added MTT reagent (tetrazolium salt) for 4 hrs at 37° C. Then the formazan product was measured spectrophotometrically at 550 nm. Higher OD value means more cell number and higher cell viability. Cell proliferation was calculated as percent of untreated control cells.

### Results

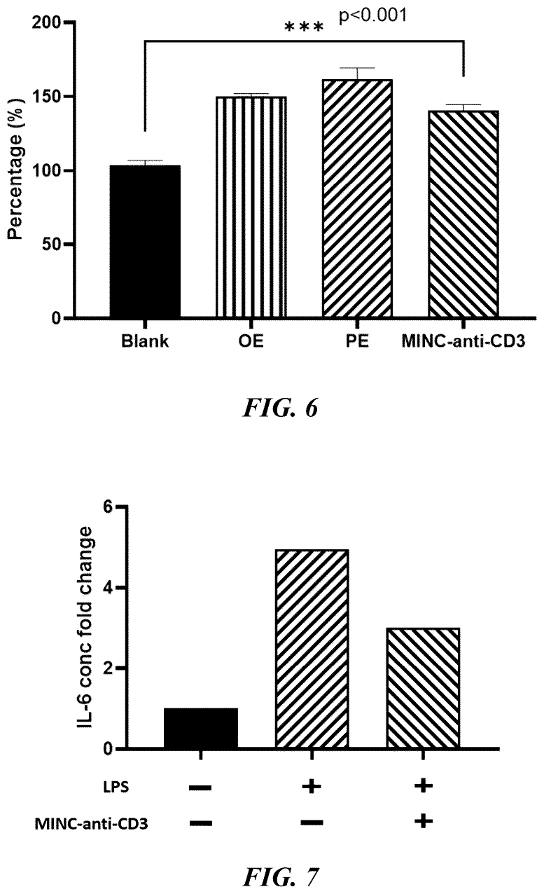

FIG. 6 shows that comparing to the saline treatment group (Blank, 100%), the cell number of OEGCG treated group increased to 150%, the cell number of PEG-ECGC treated increased to 170%, and the cell number of MINC-anti-CD3 increased to 140%. These results confirm that the ability of OEGCG, PEG-ECGC and MINC-anti-CD3 promote the proliferation/regeneration of neuron cells.

Damage of neuron cells directly causes the loss of cognitive ability of patients. This is one of the common symptoms in Alzheimer's disease, Parkinson's disease, Huntington's disease and Lewy body dementia. Currently, there is no medicine to increase the neuron cell number upon neuron cell damage. Data from this example shows that MINC-anti-CD3 stimulated the proliferation/regeneration of neuron cells, which can be applied to various neurodegenerative diseases.

### Example 7: MINC-anti-CD3 Polarizes Microglia from M1 to M2

### Materials

OEGCG and PEG-EGCG were prepared according to Example 1.

Recombinant Aβ (1-42) peptide, MTT (3-(4,5-Dimethylthiazol-2-yl)-2,5-Diphenyltetrazolium Bromide) were purchased from Abcam.

HT-22 cell was obtained from Millipore (Bedford, MA, USA).

MINC-anti-CD3 was prepared according to Example 1.

### Method

LPS was used to stimulate microglia to proinflammatory M1 phenotype (IL-6 secretion is M1 phenotype). MINC-anti-CD3 was used to reduce proinflammatory M1 phenotype of microglia.

In brief, BV-2 cells were seeded at 10000 cells per well in 96 well plate and incubated for 16 hours at 37° C. The cells were then treated with LPS (100 ng/mL) or LPS+MINC-anti-CD3 (10 μg/mL) for 24 hours. After treatment, medium supernatant was collected for IL-6 ELISA assay. The results were normalized to untreated cell control group. Cell viability of each group was also measured using MTT assay.

### Results

LPS polarized microglial cells towards proinflammatory M1 phenotype, this was indicated by the secretion of IL-6. FIG. 7 shows that after treated with MINC-anti-CD3, the amount of IL-6 was decreased, suggesting a reduced microglia M1 phenotype and implying a shift towards M2 phenotype, which is anti-inflammatory, neuron protective microglia phenotype (representative study was used for fold change calculation). We also conducted MTT assay in all groups of cells, the data showed no difference in cell number in the MINC-anti-CD3 treated group. The results indicate that MINC-anti-CD3 decreased IL-6 secretion instead of reducing cell viability (cell number).

Clinical data shows that the phenotypes of microglia in the brain is critical to disease progression in neuron degenerative diseases such as Alzheimer's disease, Parkinson's disease, Huntington's disease and Lowey body dementia. M1 microglia releases inflammatory cytokines (such as IL-6, IL-1β and TNF-α) and has neurotoxicity, while M2 microglia releases anti-inflammatory mediators (such as IL-4, IL-10 and TGF-β) and has neuroprotectivity. M2 microglia can also uptake and remove abnormal protein deposits and protect neuron cells from damage. Microglia are mostly polarized to M1 type in the brain of neurodegenerative diseases. This example shows that MINC-anti-CD3 can treat neurodegenerative diseases via reducing M1 stage microglia. The reversion of inflammation in the brain after MINC-ant-CD3 treatment is indicated.

### Example 8: MINC-Anti-CD3 Decreases the Toxicity of Anti-CD3 and Rescues the Body and Survival Rate of Animals

### Materials

Anti-CD3 was purchased from Biolegend.

MINC-anti-CD3 was prepared according to WO2011/112156.

BALB/c mice were purchased from BioLesco.

### Method

35 male 6-8 weeks Balb/c mice were kept in a specific pathogen-free environment. After 3 days acclimation, we started the treatment on the day after baseline calculation, which was designated as day 0. The free anti-CD3 antibody or MINC-anti-CD3 were administrated to mice at dose of 5, 25 and 125 μg/mouse. i.v. daily for 2 days. The mice were randomly assigned to one of three treatment groups: 1) saline; 2) anti-CD3; 3) MINC-anti-CD3, n=5. Body weight, survival rate and clinical signs were daily measured every other day for consecutive eight days. Statistical analysis was perform using GraphPad Prizm. Two-Way ANOVA is used to evaluate differences between the groups, with p<0.05 considered significant.

### Results

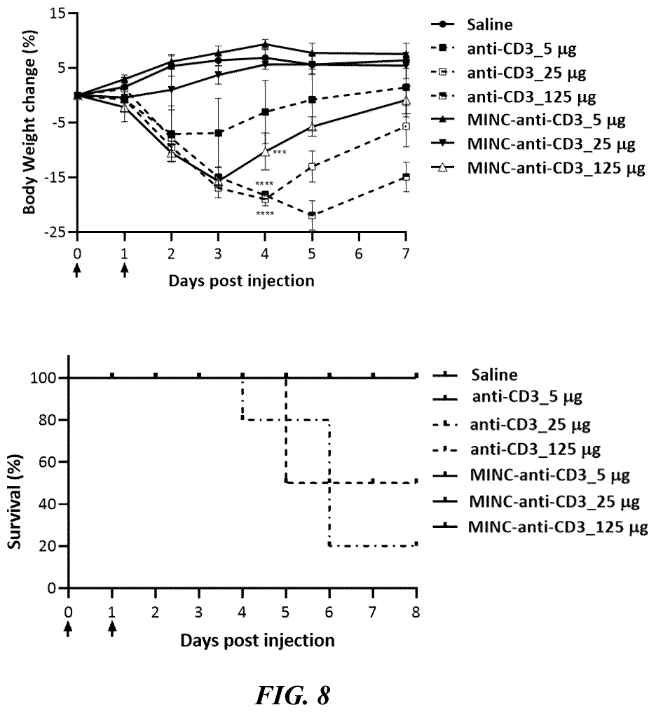

FIG. 8 shows that the injection of free anti-CD3 was toxic at all three dosages and the body weight of animal not only significantly decreased, and did not recover back to normal range. The animal body weight decreased dramatically after day 0 and day 1 injection, even in the lowest 5 μg of free anti-CD3 injection group. The loss of the body became irreversible and in the 125 μg/mouse treated group. In comparison, there was no significant change of body wight after 5 μg and 25 μg of MINC-anti-CD3 treatment. Even at the highest dosage of 125 μg, the animal body weight recovered to a comparable level as saline treated control group.

The survival rate of animal treated with free anti-CD3 treated group (125 μg) decreased at day 4 (animal death occurred), while MINC-anti-CD3 remained 100% though the end of the experiment.

The result demonstrates that MINC encapsulation significantly reduced toxicity of free anti-CD3 and was well tolerated at the dose up to 25 folds of clinically relevant dosage (5 ag/mouse). Because of the low toxicity of MINC-anti-CD3, it can be repeatedly treating patients to help patients to regain cognitive ability after long term treatment.

### Example 9: MINC-Anti-CD3 is Safe Indicated by Animal Blood Cell Analysis

### Materials

Anti-CD3 was purchased from Biolegend.

MINC-anti-CD3 was prepared according to Example 1.

C57BL/6 mice were purchased from National Laboratory Animal Center (NLAC).

### Method

14 weeks old C57BL/6 mice was tail vein i.v. injected once per day for two days with control (saline), 5 μg or 25 μg of STM-003 (in 50 μL saline). Complete blood count was analyzed at Day 4 after injection. Red blood cell (RBC), platelet (PLT), white blood cell (WBC), neutrophil (NEUT) and lymphocyte (LYMPH) were compared among the three groups.

### Results

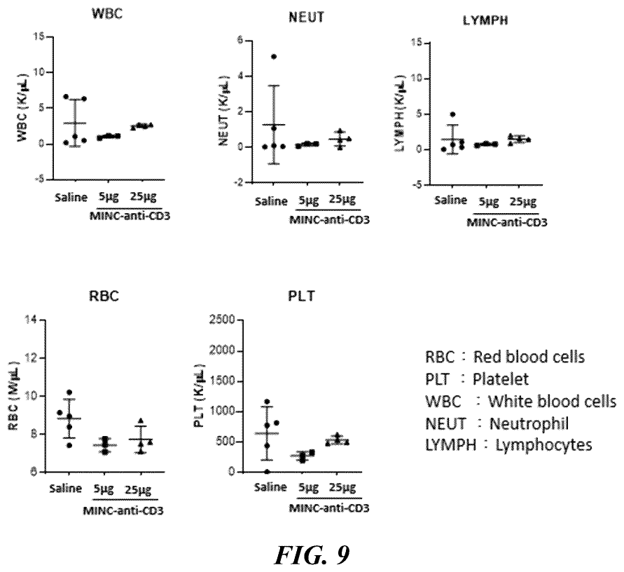

FIG. 9 shows that MINC-anti-CD3 treatment was safe in animals and did not alter the blood cell composition comparing with saline treated control group.

Past clinical studies have shown that cytokine release syndrome (CRS) often occurred in free anti-CD3 antibody treated patients. CRS majorly results from increased white blood cells in patient's blood. We did not observe significant changes of cell counts in white blood cells, red blood cells and platelets, which further proves the safety of MINC-anti-CD3 treatment. Because of the safety of MINC-anti-CD3, it can be applied for long term treatment (repeat dosing) for neurodegenerative diseases including Alzheimer's disease, Parkinson's disease, Huntington's disease and Lewy body dementia, to prevent the relapses of the disease.

### Example 10: MINC Platform Delivers Anti-CD3 and Anti-CD33 Antibody Drugs Through Blood Brain Barrier (BBB) in Surrogate Transwell Cell Model. (Prophetic Example)

### Objective

This example is to demonstrate MINC platform carries antibody agents to penetrate BBB into brain. The in vitro surrogate transwell cell model is to simulate the tight cell junctions in BBB. Compared to agents without MINC formulation, MINC-anti-CD3 or MINC-agents are expected to have stronger fluorescence intensity, which mean an increase in the amount of agents passing the BBB structure.

Anti-CD3 and anti-CD33 can be replaced by other antibodies targeting T cells and microglia in the brain including anti-CD39, anti-CD73, anti-PD-L2, anti-CTLA-4, anti-GZM A, anti-GZM-B, anti-CD33, anti-TAM, anti-FcγRI, anti-CD36, anti-RAGE, anti-APOE, anti-CR1 and anti-CD38. These antibodies are labeled with Cy5.5 following the manufacturer's instructions, and are encapsulated into MINC platform using the method described in active ingredient section.

### Materials

Anti-CD3-Cy5.5 and anti-CD33-Cy5.5 conjugate are prepared by reacting the anti-CD3 antibody and anti-CD33 with Cy5.5-NHS ester according to manufacturer's instructions.

MINC-anti-CD3, MINC-anti-CD33 and other MINC nanoparticles are prepared according to WO2011/112156.

### Method

To confirm the efficacy of MINC platform in bringing drugs across blood brain barrier, we select MINC-anti-CD3 and MINC-anti-CD33 as examples using in vitro BBB transwell study. The details are described below.

In brief, bEnd.3 or Caco-2 cells are seeded onto the luminal side of the insert of 12 well transwell plate (Falcon). TEER value of each well is measured by a Millicell ERS Voltohmmeter (Millipore, MA, USA). When the TEER value of each insert reach over 200 Ω*cm², either unencapsulated free drugs or MINC-encapsulated drugs (n=3) are added to the top of inserts, and the basolateral medium is collected at 3, 6, 12 and 24 h. The medium of upper (before penetration) and lower well (after penetration) in each treatment group is used for quantitating the drugs penetrating through BBB using fluorescence intensity, ELISA method, spectrum photometry or mass spectrum.

### Example 11: MINC Platform Delivers Anti-CD3 to the Brain in Mouse. (Prophetic Example)

### Objective

This example demonstrates MINC-anti-CD3 is delivered into brain of a mice. The in vivo mouse model is used to measure the amount of fluorescence labeled anti-CD3 in the brain area. Comparing to anti-CD3 without MINC formulation, we expect that MINC-anti-CD3 increase BBB penetration compared to the unencapsulated drug alone.

### Materials

MINC-anti-CD3-Cy5.5 is anti-CD3-Cy5.5 encapsulated within PEG-EGCG and OEGCG, and it is prepared according to Example 4.

### Method

We use fluorophore labeled anti-CD3 as an example to demonstrate that MINC formulation can bring antibody drugs to the brain in mouse model.

Athymic Nude-Foxn1^{nu} female mice at the age of 6 weeks are used, the mice are divided into two groups. One group (n=3) received 10 mg/kg anti-CD3-Cy5.5 through tail vein i.v. bolus as control. The other group (n=3) receive equivalent anti-CD3-Cy5.5 dose in MINC-anti-CD3-Cy5.5 via tail vein i.v. bolus. After drug administration, the live images are observed at 8 hours, with ex/em of 674/692 nm, using an IVIS (in vivo imaging system) Lumina III XRMS.

### Example 12: Method for Preparing MINC-Anti-CD3 Using Different Flavonoids in Flavonoid Oligomers and Polymer-Flavonoid Conjugates. (Prophetic Example)

### Objective

This example demonstrates MINC platform can use different flavonoids to encapsulate anti-CD3 and formulate nanoparticles. EC, ECG and EGCG are used to form oligomer of flavonoids or conjugated to PEG to form PEG-EC, PEG-ECG and PEG-EGCG. DLS is used to demonstrate the success of different flavonoids to form nanoparticles.

### Method

MINC anti-CD3 nanoparticles are prepared according to WO2009/054813. In brief, Different flavonoid oligomer including OEGCG or OECG is added to anti-CD3 in PBS, followed by adding different polymer-flavonoid including PEG-EGCG, PEG-ECG or PEG-EC. After incubating the mixture at room temperature, 10K MWCO centrifugal filter is used to remove the unreacted oligomer flavonoid and polymer-flavonoid. DLS (Anton Paar Litesizer 500) is used to measure the nanoparticle size.

### Example 13: Method for Preparing MINC-Anti-CD3 Using Different Hydrophilic Polymers in Polymer-Flavonoid Conjugates. (Prophetic Example)

### Objective

This example demonstrates MINC platform can use different hydrophilic polymers to encapsulate anti-CD3 and formulate nanoparticles. PEG, HA and Dextran are used to conjugated to EGCG to form PEG-EGCG, HA-EGCG and Dextran-EGCG. DLS are used to demonstrate the success of different polymers to form nanoparticles.

### Materials

OEGCG is oligomerized EGCG, which is prepared according to WO2006/124000.

PEG-EGCG is PEG conjugated with one or two EGCG. HA-EGCG is HA conjugated with one or two EGCG. Dextran-EGCG is Dextran conjugated with one or two EGCG. These different polymer-flavonoids are prepared according to WO2006/124000, WO2009/054813, or WO2015/171079.

Anti-CD3 is purchased from Biolegend.

### Method

MINC (Multi-target Immune Nanocarrier Combination)-anti-CD3 nanoparticles are prepared according to WO2009/054813. In brief, anti-CD3 is incubated in PBS. Subsequently, OEGCG or OEGCG is added to anti-CD3, followed by adding different polymer-flavonoid including PEG-EGCG, HA-EGCG and Dextran-EGCG. After incubating the mixture at room temperature, 10K MWCO centrifugal filter is used to remove the unreacted OEGCG and polymer-flavonoid. DLS (Anton Paar Litesizer 500) is used to measure the nanoparticle size.

### Example 14: MINC-Anti-CD3 Study in Alzheimer's Disease Animal Model. (Prophetic Example)

### Objective

This example demonstrates the efficacy of MINC-anti-CD3 in treating Alzheimer's disease. The brain Aβ content and behavior studies of mice or rats treated with vehicle, anti-CD3 or MINC-anti-CD3 present the improvement of spatial working memory and exploratory activity.

### Materials

OEGCG and PEG-EGCG are prepared according to Example 1.

Anti-CD3 is purchased from Biolegend.

MINC-anti-CD3 is prepared according to Example 1

### Method

In brief, Tg APPsw mice, APP/PS1 mice or Wistar rats are used. The mice or rats are divided into several groups. Each group is i.v. injected with vehicle (PBS or saline as no treatment control), anti-CD3, or MINC-anti-CD3 at equivalent anti-CD3 concentration between 0.1 to 100 mg/kg twice per week for 2-3 months, respectively. Anti-β amyloid is at a concentration ranging from to 0.01-10 μg/mL. These mice or rats are sacrificed at 6-24 months of age for analyses of Aβ levels and Aβ load in the brain. Quantitative Aβ image analysis is performed using anti-β-Amyloid (clone 4G8).

To measure spatial working memory and exploratory activity, the Morris Water Maze (MWM) test is performed. An open—field water—maze procedure in which mice learn to escape from opaque water onto a hidden platform is an established model for testing cognitive functions in mice. Spatial memory formation and retention are assessed using the MWM assay. A 10 cm escape platform is submerged 1 cm below the water surface into a circular plastic pool filled up with opaque water. Three visual cues are positioned on the walls around the pool. A digital camera is installed above the center of the maze. Images are acquired and transmitted to a PC running the tracking software. On the first three days (pre-training), the mice are trained using a visible platform (the platform is placed above the water surface). To assess spatial memory formation, the mice are trained to locate the hidden platform for 8 consecutive days. The escape latency of each trial (4 per day with an interval of 3-5 min) is recorded and analyzed by the tracking software. At training day 3 and at day 9, memory retention is assessed in a probe trial performed by removing the platform and analyzing the search pattern used by each mouse for a fixed time of 45 seconds. The escape latency which describes the time mice need to find the hidden platform is measured and analyzed as the mean value of the four trials each day.

### Example 15: MINC-Anti-CD33 Study in Alzheimer's Disease Animal Model. (Prophetic Example

### Objective

This example demonstrates the efficacy of MINC-anti-CD33 in treating Alzheimer's disease. The brain Aβ content and behavior studies of mice or rats treated with vehicle, anti-CD33 or MINC-anti-CD33 can present the improvement of spatial working memory and exploratory activity. This example is expected demonstrate MINC-anti-CD33 have therapeutic efficacy for treating Alzheimer's disease.

### Materials

OEGCG and PEG-EGCG are prepared according to Example 1.

Anti-CD33 is purchased from Biolegend.

MINC-anti-CD33 is prepared according to Example 1

### Method

To confirm the efficacy of MINC-anti-CD33 in reducing Aβ accumulation or restoring β-amyloid-induced behavioral derangements, an in vivo Alzheimer's disease model is used.

In brief, Tg APPsw mice, APP/PS1 mice or Wistar rats are used. The mice or rats are divided into several groups. Each group is i.v. injected with vehicle (PBS or saline as no treatment control), anti-CD33, or MINC-anti-CD33 at equivalent anti-CD33 concentration between 0.1 to 100 mg/kg twice per week for 2-3 months, respectively. Anti-β amyloid is at a concentration ranging from to 0.01-10 μg/mL. These mice or rats are sacrificed at 6-24 months of age for analyses of Aβ levels and Aβ load in the brain. Quantitative Aβ image analysis is performed using anti-β-Amyloid (clone 4G8).

To measure spatial working memory and exploratory activity, the Morris Water Maze (MWM) test is performed. An open-field water-maze procedure in which mice learn to escape from opaque water onto a hidden platform is an established model for testing cognitive functions in mice. Spatial memory formation and retention are assessed using the MWM assay. A 10 cm escape platform is submerged 1 cm below the water surface into a circular plastic pool filled up with opaque water. Three visual cues are positioned on the walls around the pool. A digital camera is installed above the center of the maze. Images are acquired and transmitted to a PC running the tracking software. On the first three days (pre-training), the mice are trained using a visible platform (the platform is placed above the water surface). To assess spatial memory formation, the mice are trained to locate the hidden platform for 8 consecutive days. The escape latency of each trial (4 per day with an interval of 3-5 min) is recorded and analyzed by the tracking software. At training day 3 and at day 9, memory retention is assessed in a probe trial performed by removing the platform and analyzing the search pattern used by each mouse for a fixed time of 45 seconds. The escape latency which describes the time mice need to find the hidden platform is measured and analyzed as the mean value of the four trials each day.

### Example 16: MINC-Anti-CD3 Study in Parkinson's Disease Animal Mode. (Prophetic Example

### Objective

This example demonstrates the efficacy of MINC-anti-CD3 to treating Parkinson's disease. Tyrosine hydroxylase (TH) is the rate-limiting enzyme for dopamine synthesis. A reduction in TH activity or expression is critical in the pathogenesis of Parkinson's disease. In the MPTP neurotoxin PD animal model, we expect that mice treated with MINC-anti-CD3 remain higher TH expression or enzymatic activity compared to the untreated control group. Therefore, this example is to indicate that MINC-anti-CD3 protects the dopaminergic neuron cell function. MINC-anti-CD3 has therapeutic efficacy for Parkinson's disease.

### Materials

MINC-anti-CD3 is prepared according to WO2009/054813.

CD3 antibody and 1-methyl-4-phenyl-1, 2, 3, 6-tetrahydropyridine (MPTP) are purchased from Genscript, Abcam, Thermo Fisher, or Sigma.

### Method

To confirm the efficacy of MINC-anti-CD3 in treating Parkinson's disease (PD), an in vivo PD model is used. In brief, C57/BL mice with MPTP-induced PD are used. The mice are divided into several groups. Each group is i.v. injected with vehicle (PBS or saline as no treatment control), anti-CD3 or MINC-anti-CD3 at equivalent anti-CD3 concentration between 0.1 to 100 mg/kg for 5 to 10 days, respectively. In the following 3-5 days, the mice are i.p. injected with 1 to 100 mg/kg MPTP. Three days after the last injection, these mice or rats are sacrificed. Striata from the second halves of mice brains are used for preparation of homogenates for tyrosine hydroxylase activity assay. Tyrosine hydroxylase protein level is analyzed using western blotting.

### Example 17: MINC-Anti-CD33 Study in Parkinson's Disease Animal Model. (Prophetic Example

### Objective

This example demonstrates the efficacy of MINC-anti-CD33 to treating Parkinson's disease. Tyrosine hydroxylase (TH) is the rate-limiting enzyme for dopamine synthesis. A reduction in TH activity or expression is critical in the pathogenesis of Parkinson's disease. In the MPTP neurotoxin PD animal model, we expect that mice treated with MINC-anti-CD33 remain higher TH expression or enzymatic activity compared to the untreated control group. Therefore, this example is expected to indicate that MINC-anti-CD33 protects the dopaminergic neuron cell function. MINC-anti-CD33 has therapeutic efficacy for Parkinson's disease.

### Materials

MINC-anti-CD33 is prepared according to WO2009/054813.

Anti-CD33 antibody and 1-methyl-4-phenyl-1, 2, 3, 6-tetrahydropyridine (MPTP) are purchased from Genscript, Abcam, Thermo Fisher, Sigma or any other suppliers.

### Method

To confirm the efficacy of MINC-anti-CD33 in treating Parkinson's disease (PD), an in vivo PD model is used. In brief, C57/BL mice with MPTP-induced PD are used. The mice are divided into several groups. Each group is i.v. injected with vehicle (PBS or saline as no treatment control), anti-CD33 or MINC-anti-CD33 at equivalent anti-CD33 concentration between 0.1 to 100 mg/kg for 5 to 10 days, respectively. In the following 3-5 days, the mice are i.p. injected with 1 to 100 mg/kg MPTP. Three days after the last injection, these mice or rats are sacrificed. Striata from the second halves of mice brains are used for preparation of homogenates for tyrosine hydroxylase activity assay. Tyrosine hydroxylase protein level is analyzed using western blotting.

What is claimed is:

1. A method of treating a neurodegenerative disease, comprising the step of administering to a subject in need thereof an effective amount a nanocomplex having (a) an outer shell comprising one or more polymer-flavonoid conjugates, (b) optionally an inner shell comprising one or more flavonoid oligomer, and (c) anti-CD3 antibody or anti-CD33 antibody encapsulated within the shell or shells;
wherein the flavonoid is EGCG, EC, EGC, or ECG, as shown in the structures below: 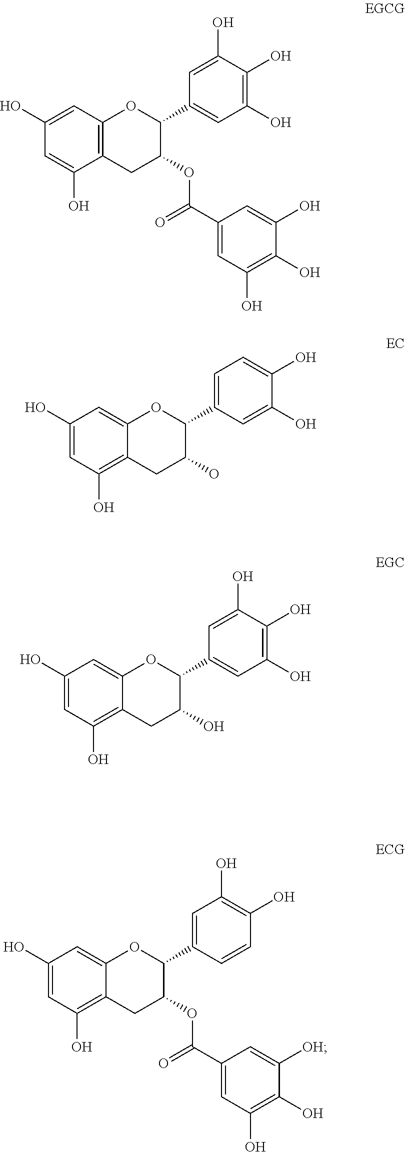 [SMILES: O=C(O[C@@H]1Cc2c(O)cc(O)cc2O[C@@H]1c1cc(O)c(O)c(O)c1)c1cc(O)c(O)c(O)c1 ; Oc1cc(O)c2c(c1)O[C@H](c1ccc(O)c(O)c1)[C@H](O)C2 ; Oc1cc(O)c2c(c1)O[C@H](c1cc(O)c(O)c(O)c1)[C@H](O)C2 ; O=C(O[C@@H]1Cc2c(O)cc(O)cc2O[C@@H]1c1ccc(O)c(O)c1)c1cc(O)c(O)c(O)c1]
the polymer is a hydrophilic polymer having a molecular weight of 1,000 to 100,000 daltons, and is selected from the group consisting of: poly(ethylene glycol) (PEG), hyaluronic acid, dextran, polyethylenimine, poloxamers, povidone, D-alpha-tocopheryl, and polyethylene glycol succinate;
the flavonoid oligomer comprises 2-20 flavonoids of EGCG, EC, EGC, or ECG;
the neurodegenerative disease is selected from the group consisting of: Alzheimer's disease, Parkinson's disease, Lewy body dementia and Huntington's disease.

2. The method according to claim 1, wherein the method reduces anti-CD3 toxicity, promotes neuron cell proliferation, prevents neuron cell death, reduce neuron oxidative stress, polarizes microglia from M1 to M2 status, and/or reduce abnormal protein aggregation.

3. The method according to claim 1, wherein the neurodegenerative disease is Alzheimer disease.

4. The method according to claim 1, wherein the neurodegenerative disease is Parkinson's disease.

5. The method according to claim 1, wherein the neurodegenerative disease is Lewy body dementia.

6. The method according to claim 1, wherein the neurodegenerative disease is Huntington's disease.

7. The method according to claim 1, wherein an anti-CD3 antibody is encapsulated within the shell or shells.

8. The method according to claim 1, wherein an anti-CD33 antibody is encapsulated within the shell or shells.

9. The method according to claim 1, wherein the flavonoid is EGCG, and the hydrophilic polymer is PEG.

10. The method according to claim 1, wherein the neurodegenerative disease is Alzheimer's disease.

11. The method according to claim 1, wherein the neurodegenerative disease is Parkinson's disease.
